(19) United States
(12) Patent Application Publication
(10) Pub. No.: US 2019/0298984 A1
(43) Pub. Date: Oct. 3, 2019

(54) TUBING SYSTEMS AND METHODS OF USE THEREOF

(71) Applicant: Carolyn H. Ethridge, Ft. Worth, TX (US)

(72) Inventor: Carolyn H. Ethridge, Ft. Worth, TX (US)

(21) Appl. No.: 16/367,211

(22) Filed: Mar. 27, 2019

Related U.S. Application Data
(60) Provisional application No. 62/648,693, filed on Mar. 27, 2018.

(51) Int. Cl. A61M 39/08; B65H 75/40; B65H 75/44

(52) U.S. Cl. CPC A61M 39/08; B65H 75/40; B65H 75/4471; A61M 16/0672; B65H 2701/33; A61M 2202/0208; B65H 75/4484

(57) ABSTRACT

A tubing system and a tubing cartridge are provided. The tubing system includes a housing having a front portion, side portions, a rear portion, a top portion, a bottom portion, and an internal portion; at least a first length of tubing positioned within the internal portion; and a first opening extending through the housing from the internal portion to an external portion thereof; wherein a first end of the first length of tubing retractably extends through the first opening. The tubing cartridge includes a body portion and at least one length of tubing substantially enclosed within the body portion, wherein the at least one length of tubing comprises a first end retractably extending through an opening in a front section of the body portion.

### RELATED APPLICATIONS

This application claims the benefit of U.S. Provisional Application Ser. No. 62/648,693, filed Mar. 27, 2018, the entire disclosure of which is incorporated herein by this reference.

### TECHNICAL FIELD

The presently-disclosed subject matter generally relates to tubing systems and methods of use thereof. In particular, certain embodiments of the presently-disclosed subject matter relate to retractable tubing systems and methods of using retractable tubing systems.

### BACKGROUND

More than 65 million people around the world have moderate or severe chronic obstructive pulmonary disease (COPD), a number experts predict will continue to rise over the next 50 years. In the United States alone, about 15 million adults have been diagnosed with COPD, while as many as 12 million more adults are believed to have undiagnosed, earlier stage COPD. This disease seriously affects not only the lives of those who are diagnosed, but the lives of their families, loved ones, and caregivers as well. It is the third most common cause of death in the US, and costs the country around $30 billion a year in healthcare expenses.

In addition to those who have COPD, about 10 million US adults have chronic bronchitis and about 5 million US adults have emphysema, the two most common conditions that contribute to COPD. Typically, treatment for individuals with COPD, chronic bronchitis, emphysema, or other diseases that affect oxygen levels (e.g., asthma, occupational lung disease, lung cancer, cystic fibrosis, congestive heart failure, etc.) includes oxygen therapy. In general, whether provided in a hospital or at home, oxygen therapy involves tubing providing a flow of oxygen directly from an oxygen source to an individual.

While oxygen therapy can provide effective treatment to those in need, existing tubing systems suffer from various drawbacks. More specifically, many systems include short lengths of tubing and/or bulky delivery systems that restrict patient movement. Other systems that include additional tubing often present a risk of tripping or falling, which can be devastating to the elderly or patients recovering from surgery. Furthermore, this tubing can become contaminated when it touches the floor, and can be very difficult to clean/replace between patients.

Accordingly, there exists a need for tubing systems that permit mobility while reducing the risk of tripping and/or cross-contamination.

### SUMMARY

The presently-disclosed subject matter meets some or all of the above-identified needs, as will become evident to those of ordinary skill in the art after a study of information provided in this document.

This summary describes several embodiments of the presently-disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this summary or not. To avoid excessive repetition, this summary does not list or suggest all possible combinations of such features.

In some embodiments, the presently-disclosed subject matter includes a tubing system including a housing having a front portion, side portions, a rear portion, a top portion, a bottom portion, and an internal portion; at least a first length of tubing positioned within the internal portion; and a first opening extending through the housing from the internal portion to an external portion thereof; wherein a first end of the first length of tubing retractably extends through the first opening. In one embodiment, the tubing system includes an access panel. In another embodiment, the access panel is movable with respect to the housing. In a further embodiment, the access panel includes an open position where the internal portion is accessible and a closed position where the internal portion is not accessible.

In some embodiments, the first length of tubing is contained within a cartridge positioned in the internal portion. In some embodiments, the cartridge is removable. In one embodiment, a second end of the first length of tubing extends through the cartridge and a second opening in the housing. In another embodiment, the second end of the first length of tubing is fluidly coupled to a fluid source, providing fluid flow through the first length of tubing. In a further embodiment, the fluid source is an oxygen source. In one embodiment, a second end of the first length of tubing is fluidly coupled to an attachment member in the cartridge. In another embodiment, the attachment member is shaped to fluidly couple with a coupling mechanism in the housing.

In some embodiments, the cartridge includes a first section having a first spool and the first length of tubing, a second end of the first length of tubing being fluidly coupled to the first spool; a second section having a second spool and a second length of tubing, a first end of the second length of tubing being fluidly coupled to the second spool; and a coupling mechanism fluidly coupling the first spool to the second spool; wherein the first spool and the second spool rotate independently of each other. In one embodiment, the first length of tubing extends and retracts independently of the second length of tubing. In one embodiment, a second end of the second length of tubing retractably extend through a second opening in the housing.

In some embodiments, the tubing system includes at least one feature on the internal portion and at least one corresponding feature on the cartridge, the at least one feature and the at least one corresponding feature detachably securing the cartridge to the housing. In some embodiments, the tubing system includes at least one of a tubing recoil mechanism and a tubing release mechanism. In some embodiments, the tubing system includes a control package that selectively dispenses and retrieves the first length of tubing.

Also provided herein, in some embodiments, is a tubing cartridge including a body portion and at least one length of tubing substantially enclosed within the body portion, wherein the at least one length of tubing comprises a first end retractably extending through an opening in a front section of the body portion. In some embodiments, the cartridge includes a spool enclosed within the body portion and an attachment member in the spool, the attachment member being sized and shaped to fluidly couple with a coupling mechanism, wherein a second end of the length of tubing is fluidly attached to the attachment member. In some embodiments, the cartridge includes at least one corresponding feature to detachably secure the cartridge to a tubing system.

Further features and advantages of the presently-disclosed subject matter will become evident to those of ordinary skill in the art after a study of the description, figures, and non-limiting examples in this document.

### BRIEF DESCRIPTION OF THE DRAWINGS

FIG. 1 shows a front view of a tubing system.

FIG. 2 shows a front perspective view of a tubing system.

FIG. 3 shows a side perspective view of a tubing system with mounting features retracted.

FIG. 4 shows a side perspective view of a tubing system with mounting features extended.

FIG. 5 shows a rear perspective view of a tubing system.

FIG. 6 shows a rear view of a tubing system.

FIG. 7 shows a front view of a tubing system without a handle.

FIG. 8 shows an exploded perspective view of a tubing system.

FIG. 9 shows a front view of a tubing system with the access panel removed.

FIG. 10 shows a front view of a tubing system with the access panel and cartridge removed.

FIG. 11 shows a perspective view of a cartridge.

FIG. 12 shows a front view of a cartridge.

FIG. 13 shows a rear view of a cartridge.

FIGS. 14A-B show attachment features on the housing (A) and cartridge (B) of a tubing system.

FIG. 15 shows a front view of an alternate attachment feature on a housing.

FIG. 16 shows a schematic view of a tension sensor according to an embodiment of the disclosure.

FIG. 17 shows a side view of a tension sensor according to an embodiment of the disclosure.

FIG. 18 shows a front view of a tubing system with a display panel.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently-disclosed subject matter. Unless defined otherwise, all technical and scientific terms used herein have the same meaning as is commonly understood by one of skill in the art to which the invention(s) belong.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9): 1726-1732).

Although any methods, devices, and materials similar or equivalent to those described herein can be used in the practice or testing of the presently-disclosed subject matter, representative methods, devices, and materials are described herein.

Following long-standing patent law convention, the terms “a”, “an”, and “the” refer to “one or more” when used in this application, including the claims.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

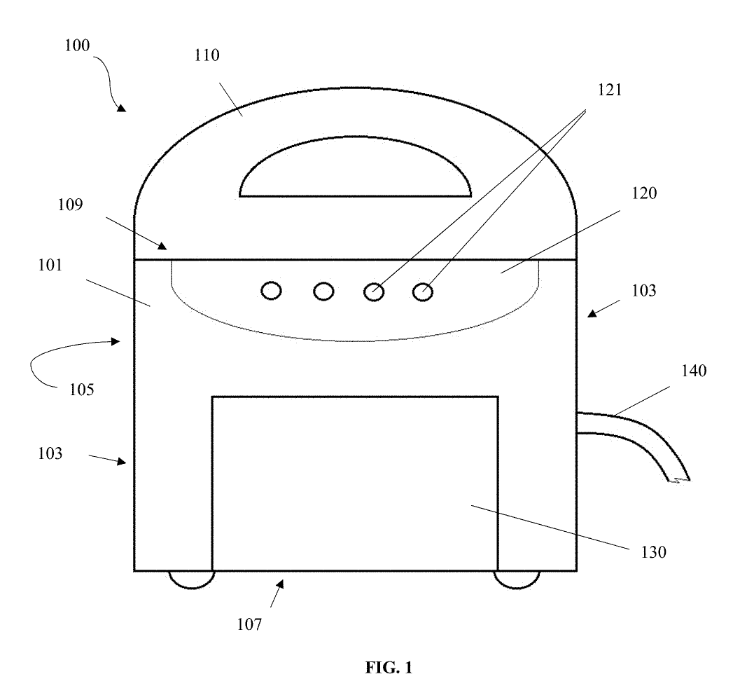

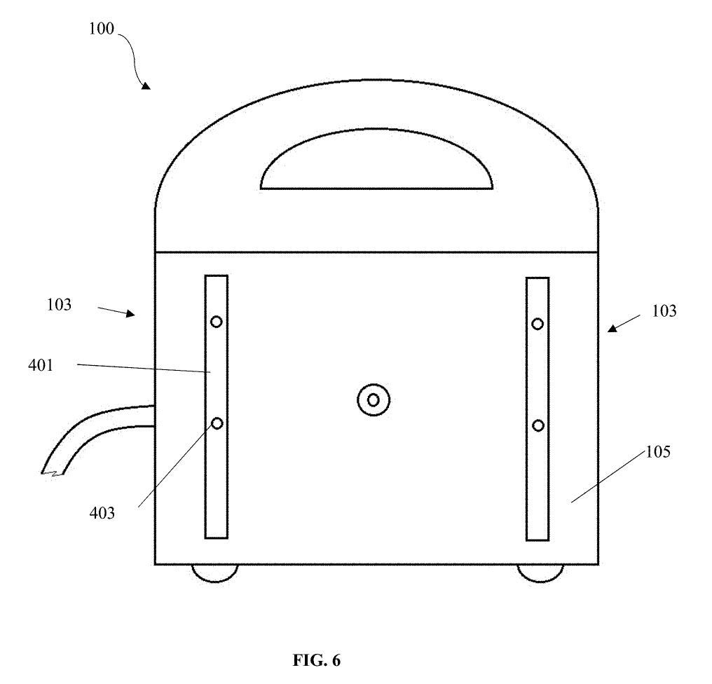

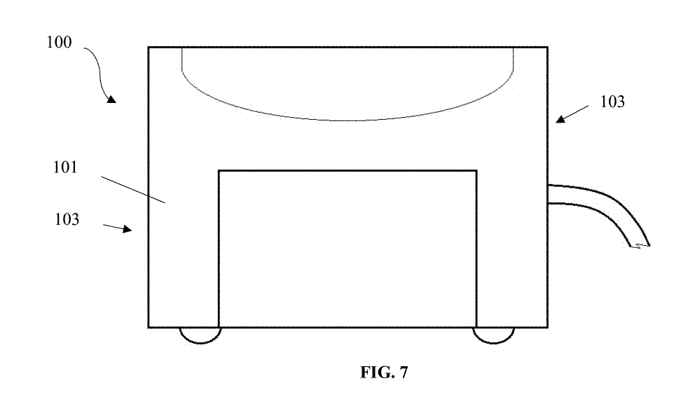

Provided herein is a tubing system for selectively dispensing and/or retrieving tubing therefrom. In some embodiments, as illustrated in FIGS. 1-7, the tubing system includes a housing 100 having a front portion 101, side portions 103, a rear portion 105, a bottom portion 107, and a top portion 109. In one embodiment, the housing 100 optionally includes a handle 110 extending from the top portion 109 thereof. FIGS. 1-6 show the housing 100 with the optional handle 110, while FIG. 7 shows the housing 100 without the optional handle 110. In another embodiment, the housing 100 includes a display/control panel 120 and/or an access panel 130.

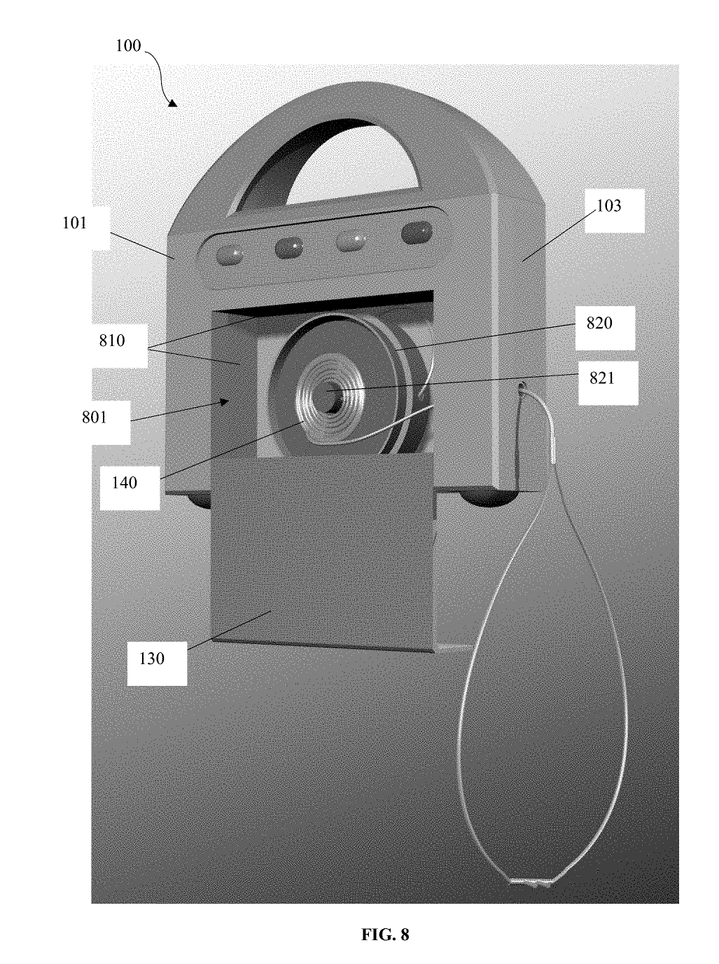

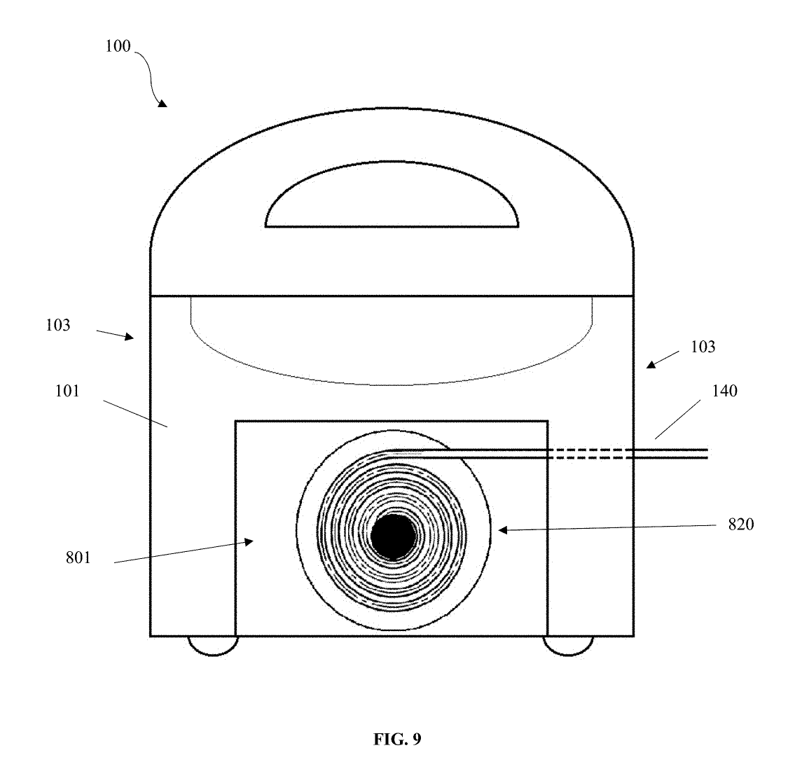

Turning to FIGS. 8-9, in some embodiments, the housing 100 includes an internal portion 801. In one embodiment, the internal portion 801 is partially defined by interior surfaces 810 of the housing 100. In another embodiment, the access panel 130 defines at least one other surface of the internal portion 801 not defined by the interior surfaces 810. In a further embodiment, the access panel 130 and the interior surfaces 810 together fully define a space enclosed by the internal portion 801. In such embodiments, the access panel 130 may be moved between an open and closed position, where the open position provides access to the internal portion 801 and the closed position inhibits access thereto.

The access panel 130 may be movable and/or removable with respect to the housing 100. For example, the access panel 130 may be secured to the housing 100 with hinges that permit movement between the open and closed positions, or the access panel 130 may be removably secured to the housing through slots and grooves, hooks and fasteners, snaps, locks, or any other suitable mechanism. Additionally, the access panel 130 may include any suitable shape and/or size for selectively providing access to the internal portion 801. For example, in one embodiment, as illustrated in FIG. 8, the access panel 130 is removable from the housing 100, and is sized and shaped to mirror sections of the front portion 101 and the bottom portion 107 not defined by the interior surfaces 810 of the housing 100.

In some embodiments, the internal portion 801 secures, holds, and/or encloses one or more lengths of tubing 140, such as, but not limited to, oxygen tubing, intravenous tubing, or any other hollow fluid conducting tubing. Referring to FIG. 8, in some embodiments, the at least one length of tubing 140 is contained within a cartridge 820 positioned in the internal portion 801 of the housing 100. The cartridge 820 includes any suitable shape and/or size for containing, dispensing, and/or retracting the at least one length of tubing 140. For example, the cartridge 820 may be round with a round spool 821 for winding and unwinding the tubing 140 around. Although described herein primarily with regard to round cartridges and spools, as will be appreciated by those skilled in the art, the disclosure is not so limited and may include any other suitable shape or combination of shapes (e.g., square or rectangular shaped cartridge with any shaped spool).

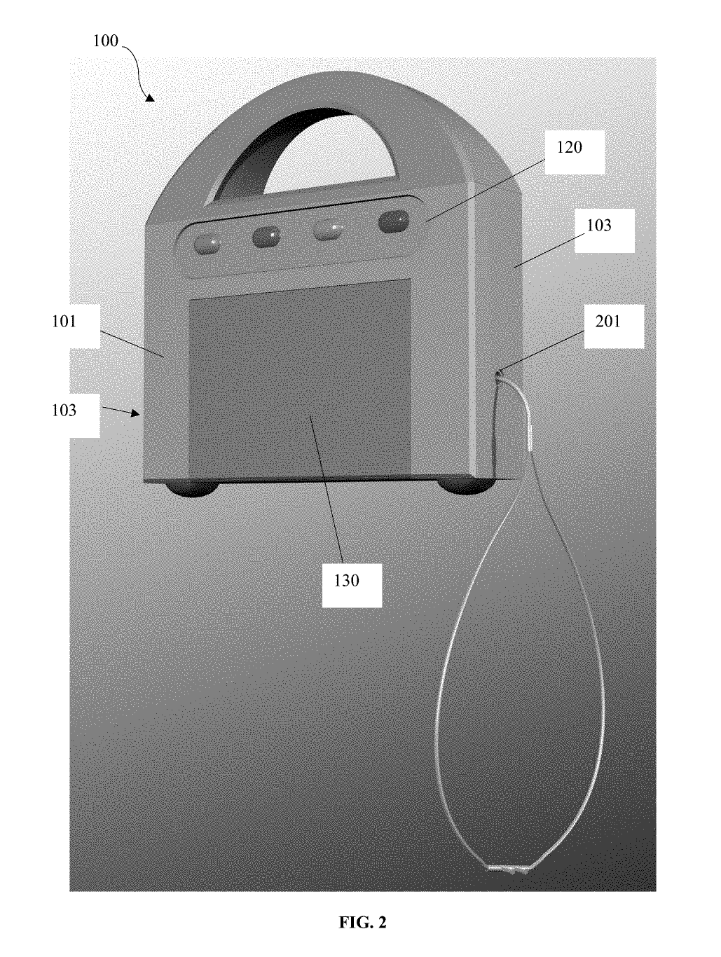

As illustrated in FIGS. 1-10, at least one end of the at least one length of tubing 140 extends from the internal portion 801 to an external portion of the housing 100. In some embodiments, as illustrated in FIG. 2, a first end of the tubing 140 passes through a first opening 201 in the side portion 103 of the housing 100. In such embodiments, the first end of the tubing 140 may be further extended and retracted from the cartridge 820 through the first opening 201, allowing the first end to move independently from the housing 100. The first end of the tubing 140 is typically a fluid outlet end of the tubing 140 (e.g., a nasal cannula containing end or other patient/user contacting end), although it is not so limited and may include a fluid inlet end (e.g., fluid source coupling end).

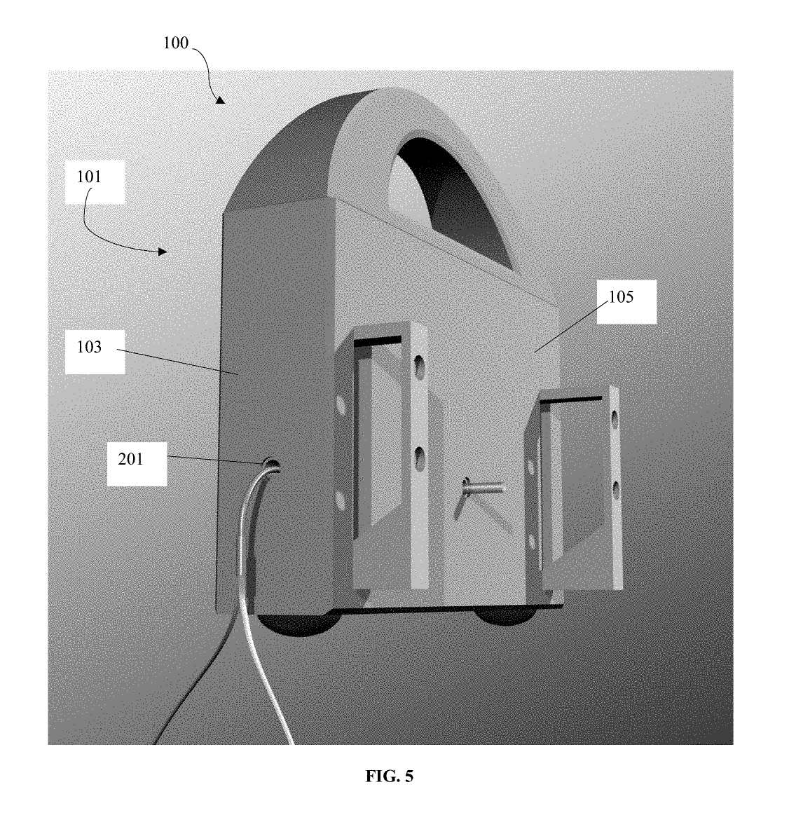

Additionally or alternatively, in some embodiments, a second end of the tubing 140 extends from the internal portion 801 to an external portion of the housing 100. For example, in one embodiment, as illustrated in FIG. 5, the second end of the tubing 140 extends from the cartridge 820 through a second opening 501 in the rear portion 105 of the housing 100. Although shown and described with the first opening 201 in the side portion 103 and the second opening 501 in the rear portion 105, as will be appreciated by those skilled in the art, the disclosure is not so limited and may include openings on any suitable side of the housing 100, including one or more openings on the same side or different sides. When the housing includes multiple openings in the side portions thereof, each opening may individually permit passage of a fluid inlet end or a fluid outlet end.

As will also be appreciated by those skilled in the art, the cartridge 820 includes any necessary openings to permit extension of the tubing 140 therethrough. For example, in some embodiments, the cartridge 820 is open on the front allowing the first end of the tubing 140 to freely extend therefrom. Alternatively, the front of the cartridge 820 may be enclosed to control the winding and unwinding of the tubing 140, and thus includes an opening in the front or side thereof for the first end of the tubing 140 to pass through. In some embodiments, the second end of the tubing extends through an opening in the spool 821 and/or the back of the cartridge 820 before passing through the second opening 501 in the housing 100. Additionally or alternatively, the cartridge 820 may include a coupling mechanism for fluidly coupling the tubing 140 thereto. In some such embodiments, the cartridge 820 includes at least two sections with independent spools that are fluidly coupled by a coupling mechanism. In one embodiment, the coupling mechanism permits at least one of the sections and/or spools to rotate independently of the other section(s) and/or spool(s) while maintaining fluid flow between separate lengths of tubing in each section. By permitting independent rotation of the different sections/spools while maintaining fluid flow between tubing, the cartridge 820 provides independent extension of the tubing 140 from each section. Accordingly, in some embodiments, the first end of the tubing 140 in the first section of the cartridge 820 may extend and retract through the first opening 201 in the housing 100, while the second end of the tubing 140 in the second section of the cartridge 820 may independently extend and retract through the second opening 501 in the housing 100, with the second end of the tubing 140 in the first section and the first end of the tubing 140 in the second section being fluidly coupled through the coupling mechanism in the spool 821.

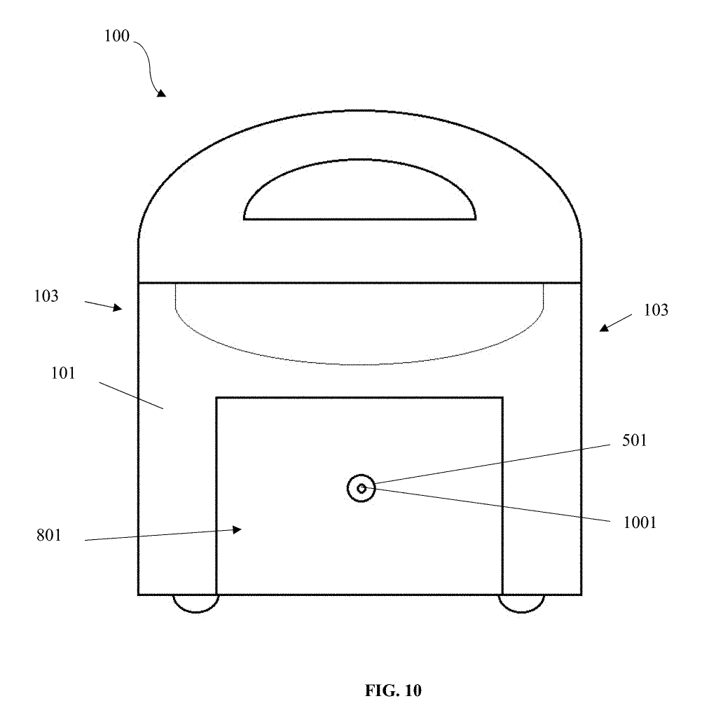

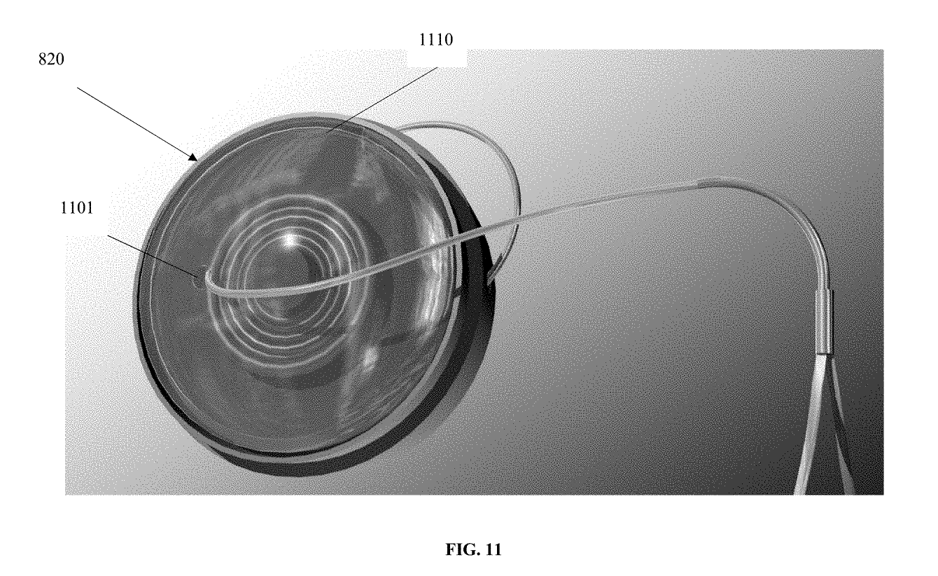

In some embodiments, as illustrated in FIGS. 10-15, the cartridge 820 is removable and/or replaceable. In some embodiments, the cartridge 820 includes a body portion substantially enclosing the at least one length of tubing positioned therein. In one embodiment, at least a first end of the tubing 140 extending through an opening 1101 in the front 1110 thereof. The cartridge 820 is positioned in the internal portion 801, and the first end of the tubing 140 is extended through the first opening 201 in the side portion 103 of the housing 100. This first end of the tubing 140 may then be extended and retracted relative to the housing 100. In some embodiments, the second end of the tubing 140 is extended through the second opening 501 in the housing 100. In one embodiment, the second end includes a fixed section of the tubing 140 extending from the cartridge 820. This fixed length is coupled to a fluid source to provide fluid flow through the length of tubing 140. In an alternate embodiment, the second end is a separately extendable section of the tubing 140, as discussed above with regard to independently rotating sections of the cartridge 820. In such embodiments, either end of the tubing extending from the housing 100 may be connected to a fluid source, with the other end providing fluid flow to a patient or desired delivery point.

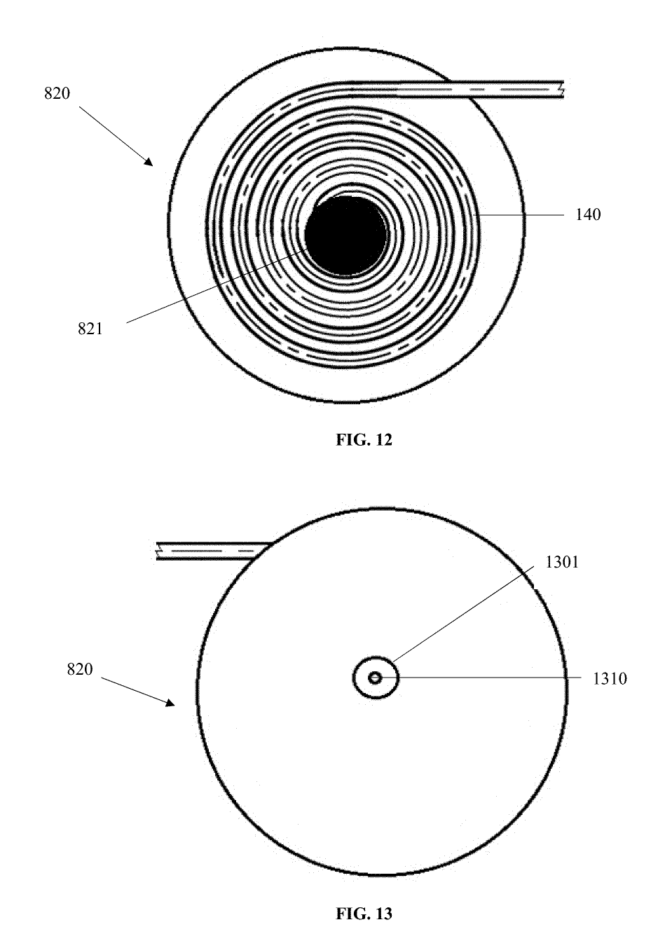

Alternatively, in some embodiments, the cartridge 820 itself or the second end of the tubing 140 may be attached to a coupling mechanism 1001 in the second opening 501. In such embodiments, as illustrated in FIG. 12, the tubing 140 either extends through the spool 821, as previously described, or is fluidly coupled to an attachment member 1310 in the spool 821. Referring to FIG. 13, on the back of the cartridge 820 is an opening 1301 that either permits the tubing 140 to pass therethrough, or includes the attachment member 1310. The attachment member 1310, when present, is arranged and disposed (e.g., sized and shaped) to fluidly couple with the coupling mechanism 1001 in the second opening 501 on the housing 100. In some embodiments, the attachment member 1310 is sized and shaped to provide a snap fit with the coupling mechanism 1001, such that when the cartridge 820 is positioned in the internal portion 801, the attachment member 1310 and the coupling mechanism 1001 mate to provide fluid communication therebetween. An additional length of tubing is attached between a second side of the coupling mechanism 1001 and a fluid source to provide fluid communication between the tubing 140 and the fluid source. Suitable fluid sources include, but are not limited to, gas sources, liquid sources, or a combination thereof. For example, suitable fluid sources may include oxygen tanks, other gaseous tanks, oxygen wall ports, other gaseous wall ports, intravenous fluid bags, or a combination thereof.

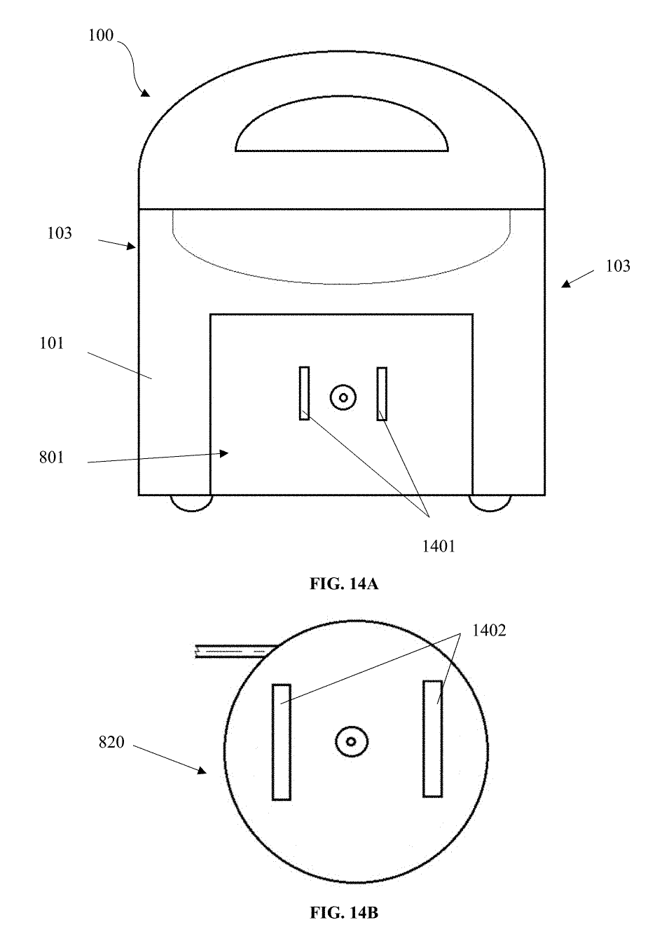

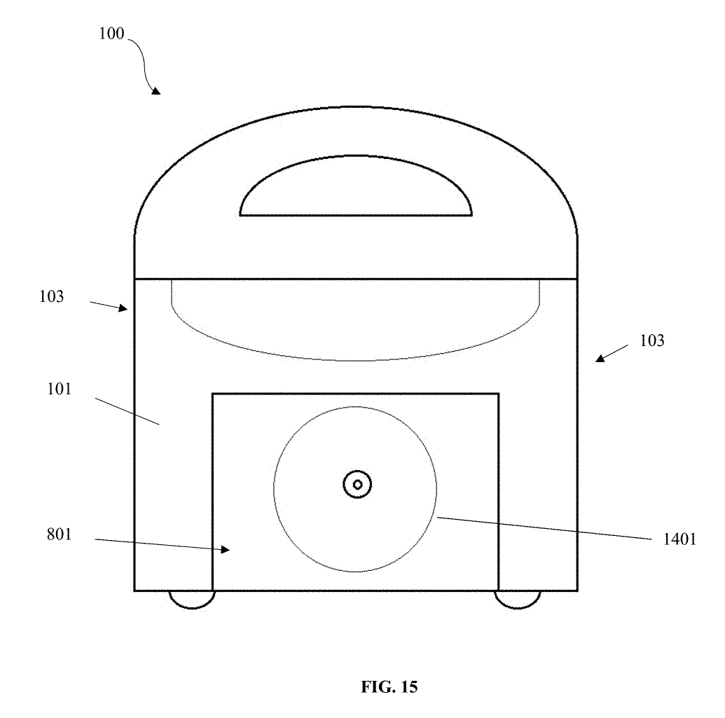

Turning to FIGS. 14A-15, in some embodiments, the internal portion 801 includes one or more features 1401 to detachably secure the at least one cartridge 820 therein. For example, the inner portion 801 may include slots, grooves, compartments, and/or any other suitable feature for orienting and/or receiving corresponding features 1402 on the at least one cartridge 820.

Once a cartridge 820 is positioned within the housing 100 and coupled to a fluid source, the tubing system provides fluid communication between the fluid source and the fluid outlet end through a retractable/dispensable length of tubing. By providing fluid flow through a retractable/dispensable length of tubing, the tubing system permits the patient/user to move away from the tubing system/fluid source without causing the tubing to touch the ground, become tangled, and/or create a tripping hazard. Additionally, by providing removable cartridges, the tubing may be quickly and easily changed between uses by removing the existing cartridge and/or inserting a new cartridge. The use of a new cartridge with fresh tubing reduces or eliminates the use of dirty/contaminated tubing from, for example, prolonged use by a single individual and/or use by a separate individual. Furthermore, in some embodiments, the cartridges include a bar code which allows the nurses to scan the cartridge into the patients' information data. This increases accuracy, accountability, and patient safety.

Although discussed above primarily with regard to a single cartridge with a single length of tubing, as will be appreciated by those skilled in the art, the disclosure is not so limited and may include additional lengths of tubing. In one embodiment, for example, the housing is arranged and disposed to receive more than one cartridge. In another embodiment, one end of the tubing from each cartridge is coupled to a fluid source, either independently or through a common port, and the other end of the tubing from each cartridge provides fluid to the patient/user. Additionally or alternatively, at least one cartridge may include more than one length of tubing contained therein. The different lengths of tubing may be connected to the same or different fluid sources, and may extend/retract at the same or different rates.

In some embodiments, the tubing system includes powered dispensing and/or retracting of the tubing 140. In one embodiment, the powered dispensing and/or retracting is controlled by a tubing recoil mechanism and/or a tubing release mechanism. The tubing recoil mechanism and the tubing release mechanism include any suitable mechanisms for extending or withdrawing the tubing, such as, but not limited to, powered rotation of the spool, a roller, a motor, a sensor, a remote, a switch, any other suitable mechanism or method, or a combination thereof. In one embodiment, for example, the tubing recoil mechanism includes a motor or spring attached to the rotating spool. In such embodiments, the motor or spring is configured to retract the tubing by rotating the spool and winding the tubing around the spool within the cartridge. In another embodiment, the tubing recoil mechanism and/or the tubing release mechanism includes one or more rollers, wheels, sheaves, pulleys, and/or other rotating elements. These rotating elements are placed in contact with the tubing such that rotation thereof dispenses and/or retracts the tubing. In a further embodiment, the tubing system includes a recoil safety mechanism such as, for example, an interrupt system and/or torque limiting system arranged and disposed to reduce or eliminate transmission or infliction of uncomfortable and/or unsafe conditions on the patient by stopping recoil of the tubing and/or activating dispensing of the tubing when elevated forces are detected on the tubing.

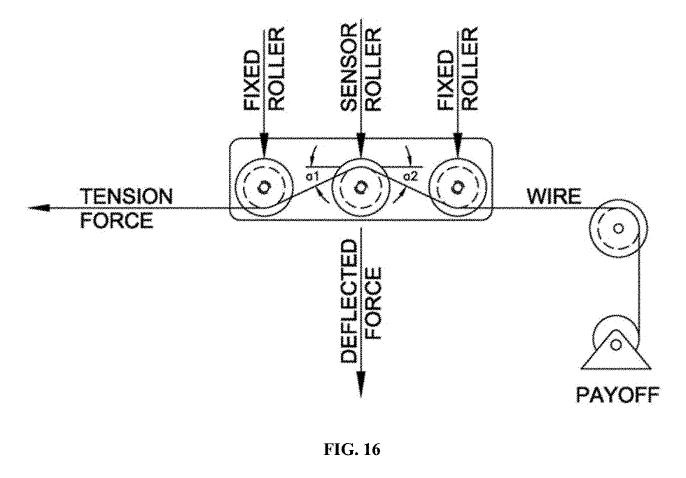

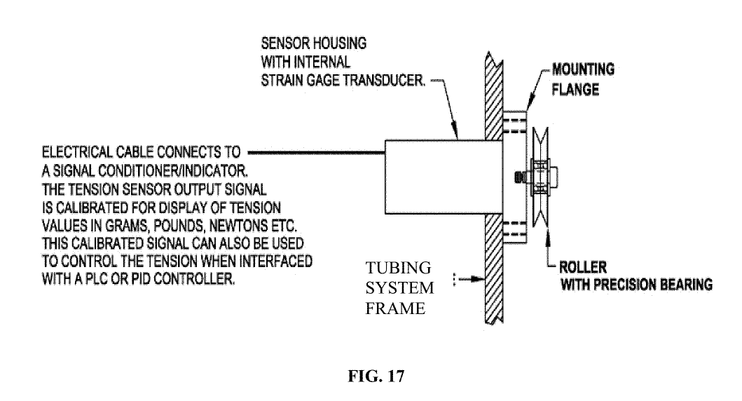

Additionally or alternatively, in some embodiments, the tubing system may include a circuit board or other control element. In one embodiment, the circuit board or other control element is coupled to the line recoil mechanism and/or the line release mechanism to control the powered dispensing and/or retracting of the tubing. In another embodiment, the circuit board or other control element is coupled to one or more sensors that provide input to automatically direct dispensing or retracting of the tubing. As illustrated in FIGS. 16-17, one such sensor includes a tension sensor assembly. Referring to FIG. 16, in one embodiment, the tubing is fed through one or more rollers, wheels, sheaves, pulleys, and/or other rotating elements arranged and disposed to dispense and/or retract the tubing by rotating. Turning to FIG. 17, in another embodiment, at least one of the rollers or other rotating elements is coupled to a tension sensor housing including a tension sensor. Together, the at least one roller or other rotating element and the tension sensor housing form an electro-mechanical assembly configured to measure the tension forces applied to the tubing. The tension sensor includes any suitable sensor, such as, but not limited to, a strain gaged transducer, or loadcell, that measures pulling force on the tubing. In certain embodiments, the longitudinal pulling force is deflected by a series of at least 3 rollers to create a fixed force vector, typically perpendicular or substantially perpendicular to the tension force vector, with the tension sensor being attached to the middle of these 3 rollers. As the tension force increases, the sensor element is deflected causing a change in the voltage output signal. This deflection may be any size depending upon the tension, the sensor, and the rotating element, but is typically small, and may include deflection of from 0.000″ to 0.004″. Upon detection of changes in tension through changes in deflection, the tension sensor electrically and/or wirelessly communicates with the circuit board or other control element, which automatically activates the line recoil mechanism and/or the line release mechanism to dispense or retract the tubing based upon the measured tension change. These components may be contained anywhere in the housing 100 and/or cartridge 820 to control dispensing and retracting of the tubing 140.

As will be appreciated by those skilled in the art, the tubing system is not limited to the tension sensor described above, and may include any other suitable sensor or combination of sensors in addition to or in place of the tension sensor. One such sensor includes a motion sensor arranged and disposed to detect movement of the patient/user. These motion sensors may independently activate dispensing and/or retracting of the tubing based upon detected movement, or may work in combination with other sensors such as the tension sensor discussed above. For example, when the motion sensor detects movement away from the tubing system it may communicate with the tension sensor to dispense the tubing at an appropriate rate. Another sensor includes a limit switch, which is arranged and disposed to sense force in the tubing and prevent the motor from recoiling when the force is at or above a certain amount. Additionally or alternatively, the tubing system may include one or more mechanisms for manually dispensing and/or retracting the tubing. For example, the control panel on the front portion of the housing may include one or more manual controls or switches for dispensing and/or retracting the tubing.

The sensors and/or switches according to one or more of the embodiments disclosed herein may communicate with the circuit board or other control element through any suitable form of communication. Suitable forms of communication include, but are not limited to, direct electrical communication, infrared, radio frequency, Bluetooth, any other suitable form of communication, or a combination thereof. In certain embodiments, where the communication includes infrared, the tubing system may also include one or more passive infrared (PIR) sensors for receiving the infrared signals. Although discussed above with regard to sensors and/or switches communicating through the circuit board or other control element, as will be appreciated by those skilled in the art, the disclosure is not so limited and may include direct communication between one or more of the sensors and/or switches and the line recoil mechanism and/or the line release mechanism.

In some embodiments, one or more of the sensors, the line recoil mechanism, and/or the line release mechanism are contained in the cartridge. In such embodiments, similar to communication between the sensors and/or switches and the circuit board or other control element, the communication between the circuit board or other control element and the cartridge may be through any suitable form of communication. In one embodiment, for example, when positioned within the internal portion the cartridge is electrically coupled to the housing through an electrical contact. This electrical coupling places the cartridge in electrical communication with the circuit board or other control element. In another embodiment, the cartridge is placed in wireless communication with the housing when positioned therein.

Regardless of the location of the one or more of the sensors, the line recoil mechanism, and/or the line release mechanism, the cartridge may still be placed in communication with the housing. Additionally, the housing and/or the cartridge may wired or wirelessly communicate with other devices. Such communication may include, but is not limited to, receiving and/or recording information; sending information; activating various mechanisms, programs, or devices; or a combination thereof. For example, the cartridge and the housing may communicate information such as date and time the cartridge was loaded and the date and time the cartridge was removed. This type of information may be used to avoid cross contamination between patients and/or extended use of tubing.

Furthermore, in some embodiments, the tubing system may include a control package for selectively dispensing or retrieving the tubing. In one embodiment, the control package includes a hand operated control using control interfaces on the display/control panel 120. In one embodiment, the control interfaces include physical controls 121, such as, but not limited to, buttons, switches, knobs, and/or other elements to operate the tubing reel assembly (e.g., the spool within the cartridge). Operation of the tubing reel assembly includes, but is not limited to, turning the tubing system on/off, retracting tubing within the housing, dispensing and/or releasing tubing from the housing, and/or activating/adjusting sensors in the tubing system. In another embodiment, the control package includes a wireless protocol such as, for example, radio frequency circuitry, infrared circuitry, Bluetooth, and/or any other suitable wireless protocol such that wiring between the control package and the tubing reel assembly may be eliminated. In an alternative embodiment, the control package may include a voice activated protocol allowing the patient and/or caregiver to control the oxygen hose reel assembly through voice commands.

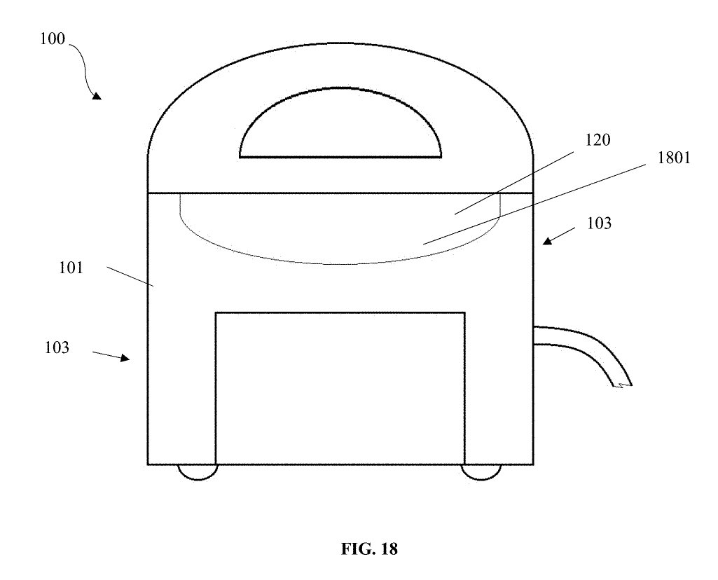

As illustrated in FIG. 18, the display/control panel 120 may also include a display screen 1801, such as a video screen. The display screen may display any suitable text, image, video, or combination thereof. Suitable displays include, but are not limited to, information about the tubing system (e.g., amount of tubing dispensed, amount of tubing available for dispensing, power status, connection status, fluid flow, fluid type, maintenance status, and/or any other information relating to the tubing system), information about the patient and/or individual using the tubing system, operating instructions, variable identifiers, any other suitable text, image, and/or video, or a combination thereof. In certain embodiments, the display/control panel includes a touch screen, providing a user with variable switches/controls on the display.

As will be appreciated by those skilled in the art, in order to provide powered dispensing and/or retracting of the tubing, the tubing system must be electrically powered. Accordingly, in some embodiments, the housing is plugged into an electrical outlet, secured to an electrically powered docking station, and/or includes a battery disposed therein. In embodiments including a battery, the battery may be rechargeable and/or may be charged when the housing is plugged into an electrical outlet. The battery provides electrical power when the housing is unplugged from an electrical outlet. In certain embodiments, the internal portion of the housing includes one or more electrical couplings that provide electrical power to the at least one cartridge when inserted and/or secured therein.

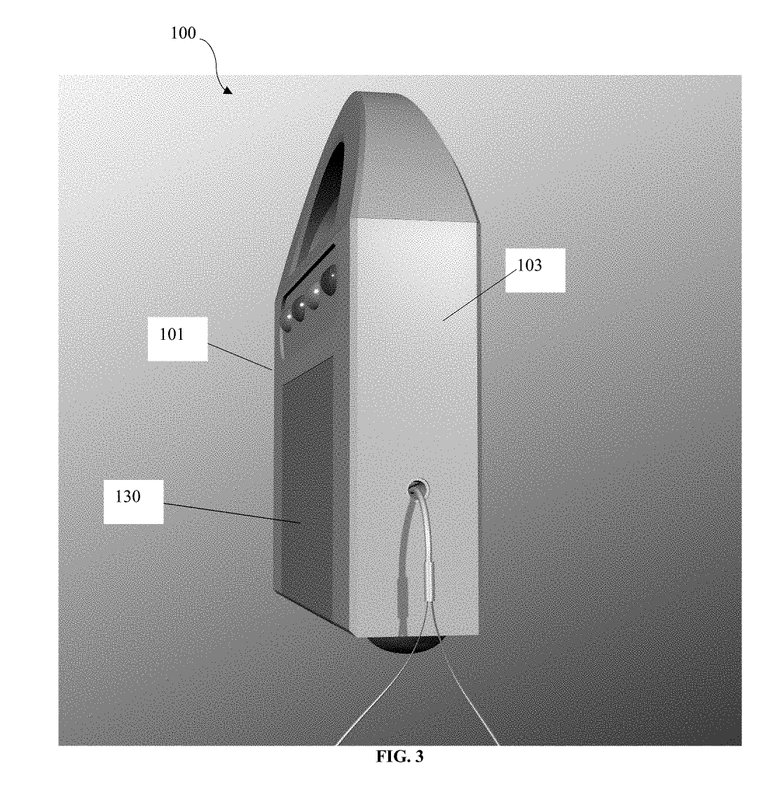

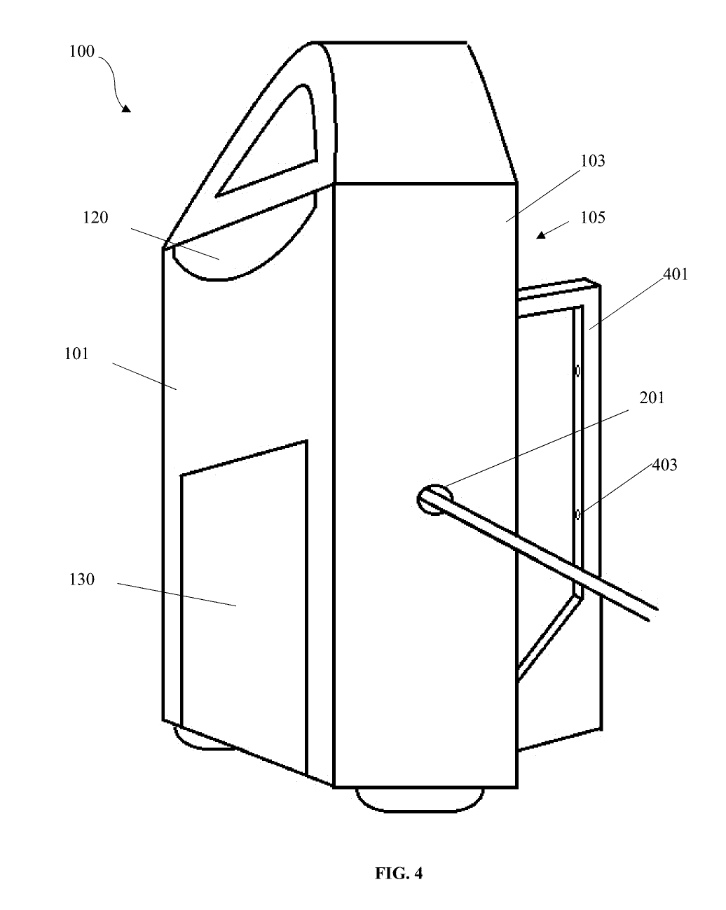

In some embodiments, the housing 100 is mountable. For example, in one embodiment, as illustrated in FIGS. 4-6, the rear portion 105 may include one or more mounting and/or supporting features 401. In one embodiment, the mounting and/or supporting features 401 include brackets. In another embodiment, the mounting and/or supporting features 401 extend from the rear portion to hold the housing at a predetermined distance from a surface upon which the housing 100 is mounted/supported. The predetermined distance includes, but is not limited to, any suitable distance for extension of tubing 140 from the rear portion 105 and/or attachment and detachment of tubing 140 from a fluid source while the housing 100 is mounted and/or supported by a mounting/supporting surface. In a further embodiment, as illustrated in FIG. 3, the mounting and/or supporting features 401 are retractable within and/or removable from the housing 100.

The mounting and/or supporting features 401 may be secured to a mounting/supporting surface through any suitable method or component. One example includes inserting fasteners (e.g., screws, nails, or other fasteners) through holes 403 formed in the mounting/supporting features 401 and affixing the fasteners to the mounting/supporting surface. Another example includes providing mounting/supporting features having shaped holes for receiving and slideably engaging projections extending from the mounting/supporting surface. Other examples include, but are not limited to, projections extending from the mounting and/or supporting features, the projections being arranged and disposed to engage brackets on the mounting/supporting surface; hook and loop fasteners; shaped edges of the mounting and/or supporting features arranged and disposed to engage brackets and/or tracks on the mounting/supporting surface, or a combination thereof. As will be appreciated by those skilled in the art, the mounting/supporting surface may be any surface or article upon which mounting/supporting of the housing would be desirable. Such surfaces include, but are not limited to, walls, beds, hospital beds, fluid tanks, fluid tank holders, moveable structures, or a combination thereof.

The tubing systems disclosed herein may be used in a hospital, care facility, and/or home setting. By retracting/extending the tubing as disclosed above, the tubing system reduces or eliminates contact between the tubing and the floor as the end user moves. This reduces or eliminates contamination of the tubing, which decreases risk of infection from the tubing. In addition, the cartridges may be removed and/or replaced between patients, which decreases the risk of cross-contamination. Furthermore, by retracting/extending the tubing as an end user moves, the tubing system reduces excess tubing outside the housing, which decreases or eliminates the risk of tripping an end user.

The presently-disclosed subject matter is further illustrated by the following specific but non-limiting examples. The following examples may include compilations of data that are representative of data gathered at various times during the course of development and experimentation related to the presently-disclosed subject matter.

### EXAMPLES

### Example 1

A tubing system is provided including a single removable cartridge. Upon insertion of the cartridge into the internal portion of the housing, the fluid inlet end of the length of tubing is passed through the opening in the rear portion of the housing and the fluid outlet end of the length of tubing is passed through the opening in the side portion of the housing. The fluid inlet end is then coupled to a fluid source, such as an oxygen tank or oxygen port. Once the fluid inlet end is coupled to an oxygen source, the fluid outlet end provides a flow of oxygen to the patient/user. As the patient/user moves away from the housing a tension sensor detects an increase in tension on the tubing. This increase in tension is communicated to a circuit board, which activates a line release mechanism to dispense more tubing. Alternatively, as the patient/user moves towards the housing, the tension sensor detects a decrease in tension on the tubing. This decrease in tension is communicated to the circuit board, which activates a line recoil mechanism to retract the tubing. By retracting the tubing the tubing system prevents the tubing from contacting the floor, thus reducing or eliminating contamination of the tubing and/or risk of tripping over the tubing.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A tubing system, comprising:
a housing including:
a front portion;
side portions;
a rear portion;
a top portion;
a bottom portion; and
an internal portion;
at least a first length of tubing positioned within the internal portion; and
a first opening extending through the housing from the internal portion to an external portion thereof;
wherein a first end of the first length of tubing retractably extends through the first opening.

2. The tubing system of claim 1, further comprising an access panel.

3. The tubing system of claim 2, wherein the access panel is movable with respect to the housing.

4. The tubing system of claim 3, wherein the access panel includes an open position where the internal portion is accessible and a closed position where the internal portion is not accessible.

5. The tubing system of claim 1, wherein the first length of tubing is contained within a cartridge positioned in the internal portion.

6. The tubing system of claim 5, wherein the cartridge is removable.

7. The tubing system of claim 6, wherein a second end of the first length of tubing extends through the cartridge and a second opening in the housing.

8. The tubing system of claim 7, wherein the second end of the first length of tubing is fluidly coupled to a fluid source, providing fluid flow through the first length of tubing.

9. The tubing system of claim 8, wherein the fluid source is an oxygen source.

10. The tubing system of claim 6, wherein a second end of the first length of tubing is fluidly coupled to an attachment member in the cartridge.

11. The tubing system of claim 10, wherein the attachment member is shaped to fluidly couple with a coupling mechanism in the housing.

12. The tubing system of claim 6, wherein the cartridge comprises:
a first section having a first spool and the first length of tubing, a second end of the first length of tubing being fluidly coupled to the first spool;
a second section having a second spool and a second length of tubing, a first end of the second length of tubing being fluidly coupled to the second spool; and
a coupling mechanism fluidly coupling the first spool to the second spool;
wherein the first spool and the second spool rotate independently of each other.

13. The tubing system of claim 12, wherein the first length of tubing extends and retracts independently of the second length of tubing.

14. The tubing system of claim 12, wherein a second end of the second length of tubing retractably extend through a second opening in the housing.

15. The tubing system of claim 6, further comprising at least one feature on the internal portion and at least one corresponding feature on the cartridge, the at least one feature and the at least one corresponding feature detachably securing the cartridge to the housing.

16. The tubing system of claim 1, further comprising at least one of a tubing recoil mechanism and a tubing release mechanism.

17. The tubing system of claim 1, further comprising a control package that selectively dispenses and retrieves the first length of tubing.

18. A tubing cartridge, comprising:
a body portion;
at least one length of tubing substantially enclosed within the body portion;
wherein the at least one length of tubing comprises a first end retractably extending through an opening in a front section of the body portion.

19. The tubing cartridge of claim 18, further comprising:
a spool enclosed within the body portion; and
an attachment member in the spool, the attachment member being sized and shaped to fluidly couple with a coupling mechanism;
wherein a second end of the length of tubing is fluidly attached to the attachment member.

20. The tubing cartridge of claim 18, further comprising at least one corresponding feature to detachably secure the cartridge to a tubing system.
